(19) United States
(12) Patent Application Publication
(10) Pub. No.: US 2022/0088124 A1
(43) Pub. Date: Mar. 24, 2022

(54) COMPOSITIONS AND METHODS FOR TREATING NECROTIZING ENTEROCOLITIS

(71) Applicant: Empire Biotechnologies, Inc., San Francisco, CA (US)

(72) Inventor: Donald Adam McCarthy, San Francisco, CA (US)

(73) Assignee: Empire Biotechnologies, Inc., San Francisco, CA (US)

(21) Appl. No.: 17/046,694

(22) PCT Filed: Apr. 11, 2019
(86) PCT No.: PCT/US19/26961

§ 371 Date: Oct. 9, 2020

Related U.S. Application Data
(60) Provisional application No. 62/656,669, filed on Apr. 12, 2018.

(51) Int. Cl. A61K 38/17; A61K 9/00; C07K 16/28; A61P 1/00

(52) U.S. Cl. CPC A61K 38/1709; A61K 9/0053; A61P 1/00; C07K 16/2842; C07K 16/2839; C07K 16/2848

(57) ABSTRACT

The invention provides compositions and methods for treating NEC by promoting weight gain and reducing intestinal inflammation by agonizing intestinal v 3 and v 5 and/or gastric 8 1 integrins. In particular, the invention relates to treating NEC with Milk Fat Globule EGF-like 8 (Mfge8) also known as human Lactadherin.

### CROSS REFERENCE TO RELATED APPLICATIONS

This application claims the benefit of U.S. Provisional Application No. 62/656,669, filed Apr. 12, 2018, which is incorporated herein by reference in its entirety.

### FIELD OF THE INVENTION

The invention provides compositions and methods for treating NEC by promoting weight gain and reducing intestinal inflammation by agonizing intestinal αvβ3 and αvβ5 and/or gastric α8β1 integrins. In particular, the invention relates to treating NEC with Milk Fat Globule EGF-like 8 (Mfge8) also known as human Lactadherin.

### BACKGROUND OF THE INVENTION

Necrotizing enterocolitis (NEC) is a devastating disease of premature infancy that affects 1-3 babies per 1000 live births. The incidence increases with low birth weight and can be as high as 12% in infants weighing less than 1.5 kg at birth. It is a major cause of morbidity and mortality and places a tremendous $1 billion per year burden on U.S. hospitals. It accounts for nearly 20% of neonatal intensive care unit costs per year. NEC prevalence and mortality have remained unchanged for years. Despite a growing understanding of the factors that predispose to NEC, there are currently no FDA-approved therapies. Treatment depends on the clinical staging of the disease and largely consists of bowel rest, discontinuation of enteral feeds, bowel decompression, and broad-spectrum antibiotics. Upon disease progression, intensive cardiovascular and respiratory support may be required along with surgical intervention. Breast milk can be protective against NEC. Probiotics have also been shown to reduce the risk of NEC but their use is controversial among neonatologists given concerns over probiotic-associated sepsis and various unknowns regarding dosage and duration of treatment.

With NEC, the intestinal wall is invaded by bacteria which causes local infection and inflammation that can ultimately destroy the bowel wall. This can lead to intestinal perforation and stool spillage into the infant's abdomen leading to an overwhelming infection and death. NEC is caused by reduced oxygen or blood flow to the intestine causing it to become weak. This weakened state makes it easier for bacteria from food entering the intestine to cause damage to the intestinal tissues. About 10% of preterm infants suffer from NEC with a mortality rate of about 30%. Patients with suspected NEC progress rapidly to severe morbidity and mortality. Despite significant efforts, no single cause has been identified. Healthy neonatal weight gain is the only determining factor for positive outcomes.

Milk fat globule-EGF factor 8 protein (Mfge8), also known as lactadherin, is a secreted breast milk protein that is encoded by the MFGE8 gene in humans. It has numberous isoforms (See, e.g. SEQ ID NOS:1-7.) MFGE8 contains a phosphatidylserine (PS) binding domain and an Arginine-Glycine-Aspartic acid motif that enables the binding to integrins. Mfge8 regulates the absorption of dietary fat by enterocytes by binding to αvβ3 and αvβ5 integrins. Integrin ligation by Mfge8 activates a PI3 kinase/mTORC2/PKCζ-dependent pathway that results in cellular uptake of free fatty acids (FFA). Mfge8 promotes nutrient absorption by slowing gastrointestinal transit time through ligation of the α8β1 integrin. Mfge8 also binds to the αvβ3 and αvβ5 integrins causing the release of FFAs from. cytoplasmic lipid droplets in enterocytes and by increasing intracellular triacylglycerol (TG) hydrolase activity.

### SUMMARY OF THE INVENTION

The invention provides compositions and methods for treating NEC by promoting weight gain and reducing intestinal inflammation by agonizing intestinal αvβ3 and αvβ5 and/or gastric α8β1 integrins. In particular, the invention relates to treating NEC with Milk Fat Globule EGF-like 8 (Mfge8) also known as human Lactadherin.

Thus, the invention provides a method for treating or preventing necrotizing enterocolitis (NEC) in a human infant, comprising orally administering an agonist for an αvβ3 □integrin receptor in intestinal enterocytes of the human infant. It also provides a method for treating or preventing necrotizing enterocolitis (NEC) in a human infant, comprising orally administering an agonist for an αvβ5 integrin receptor in intestinal enterocytes of the human infant. It also provides a method for treating or preventing necrotizing enterocolitis (NEC) in a human infant, comprising orally administering an agonist for an α8β1 integrin receptor in antral smooth muscle tissue of the human infant.

In some embodiments, the agonist is an isolated milk fat globule-EGF factor 8 (Mfge8) protein having Mfge8 or lactadherin activity. In a preferred embodiment, the Mfge8 has at least a 90% sequence identity to any one of SEQ ID NOS:1-7. In a most preferred embodiment, the Mfge8 has the sequence of any one of SEQ ID NOS:1-7.

In some embodiments, the Mfge8 is administered at a dosage of between about 0.001 and 0.5 mg/kg of body weight. In preferred embodiments, the dosage is between about 0.005 and 0.05 mg/kg of body weight or 0.01 and 0.05 mg/kg of body weight. In other embodiments, the dosages are about 0.05 mg/kg, about 0.10 mg/kg, or about 0.50 mg/kg of body weight.

In some embodiments, the agonist comprises an immunoglobulin domain. In other embodiments, the agonist comprises an immunoglobulin A (IgA) domain. In other embodiments, the agonist comprising the immunoglobulin domain further comprises Mfge8.

In preferred embodiments, the agonist is an antibody that binds to the integrin receptors having an equilibrium dissociation constant (K_{D}) of ≤1 pM, ≤10 pM ≤100 pM, ≤1 nM, ≤10 nM, or ≤100 nM. In a preferred embodiment, the antibody is a monoclonal antibody. In a more preferred embodiment, the antibody is a human monoclonal antibody. In another more preferred embodiment, the antibody is a humanized monoclonal antibody.

In some embodiments of the invention, the agonist is administered in a capsule, tablet, gel, or liquid formulation.

The invention provides a use of an agonist for an αvβ3, αvβ5, or □α8β1 integrin receptor for treating necrotizing enterocolitis (NEC) in a human infant. In some embodiments of the use, the agonist comprises an isolated milk fat globule-EGF factor 8 (Mfge8) protein having Mfge8 or lactadherin activity.

### BRIEF DESCRIPTION OF THE DRAWINGS

FIG. 1 shows that TNBS was used to generate a mouse model of NEC. It resulted in about a 17% weight loss in mice over the course of 24 days. Recombinant Mfge8 (rMfge8) at 0.05 and 0.10 mg/kg rescued the weight loss.

FIG. 2 shows that the TNBS induced NEC reduces overall survival of newborn mice to 27.3% after 6 days. Both dosages increased survival to 55.6%.

### DETAILED DESCRIPTION OF THE INVENTION

Mfge8 increases absorption of ingested nutrients by promoting enterocyte fatty acid absorption and utilization. It is also a potent anti-inflammatory molecule via its role in promoting apoptotic cell clearance and inhibition of toll like receptor (TLR) signaling. Thus, the invention provides compositions and methods for treating NEC by promoting weight gain and reducing intestinal inflammation by agonizing intestinal αvβ3 and αvβ5 integrins. In particular, the invention relates to treating NEC with Mfge8, also known as lactadherin.

An “agonist” is a substance that binds to a receptor and activates the receptor to produce a biological response. They can be in the form of antibodies, antigen-binding fragments, proteins, peptides, glycoproteins, glycopeptides, glycolipids, polysaccharides, oligosaccharides, nucleic acids, bioorganic molecules, peptidomimetics, chemicals, pharmacological agents and their metabolites, and the like. In contrast, an “antagonist” refers to a molecule capable of neutralizing, blocking, inhibiting, abrogating, reducing or interfering with the activities of a particular or specified protein, including its binding to one or more receptors in the case of a ligand, or binding to one or more ligands in case of a receptor.

“Antibodies” (Abs) and “immunoglobulins” (Igs) refer to glycoproteins having similar structural characteristics. While antibodies exhibit binding specificity to a specific antigen, immunoglobulins include both antibodies and other antibody-like molecules that generally lack antigen specificity. Polypeptides of the latter kind are, for example, produced at low levels by the lymph system and at increased levels by myelomas.

An “effective amount” refers to an amount of therapeutic compound that is effective, at dosages and for periods of time necessary; to achieve the desired therapeutic or prophylactic result. A “therapeutically effective amount” of a therapeutic compound may vary according to factors such as the disease state, age, sex, and weight of the individual. A therapeutically effective amount may be measured, for example, by improved survival rate, more rapid recovery, or amelioration, improvement or elimination of symptoms, or other acceptable biomarkers or surrogate markers. A therapeutically effective amount is also one in which any toxic or detrimental effects of the therapeutic compound are outweighed by the therapeutically beneficial effects. A “prophylactically effective amount” refers to an amount of therapeutic compound that is effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

“Homologs” are bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a preferred embodiment, homologous or derivative sequences share at least an 80 or 85 percent sequence identity. In a more preferred embodiment, homologous or derivative sequences share at least an 86, 87, 88, 89, 90, 91, 92, 93, 94, 95, 96, 97, 98, or 99 percent sequence identity. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

An “individual,” “subject” or “patient” is a vertebrate. In certain embodiments, the vertebrate is a mammal. Mammals include, but are not limited to, primates (including human and non-human primates) and rodents (e.g., mice, hamsters, guinea pigs, and rats). In certain embodiments, a mammal is a human. A “control subject” refers to a healthy subject who has not been diagnosed as having a disease, dysfunction, or condition that has been identified in an individual, subject, or patient. A control subject does not suffer from any sign or symptom associated with the disease, dysfunction, or condition.

As used herein, an antibody “interacts with” an integrin when the equilibrium dissociation constant (KD) is equal to or less than 5 nM, preferably less than 1 nM, preferably less than 100 pM, preferably less than about 50 pM, more preferably less than about 20 pM, most preferably less than about 10 pM, more preferably less than about 5 pM, yet more preferably less than about 2 pM. The term “dissociation constant” is sometimes used interchangeably with “equilibrium dissociation constant.” It refers to the value obtained in a titration measurement at equilibrium, or by dividing the dissociation rate constant (koff) by the association rate constant (kon). The association rate constant, the dissociation rate constant and the equilibrium dissociation constant are used to represent the binding affinity of an antibody to an antigen. Methods for determining association and dissociation rate constants are well known in the art.

A “medicament” is an active drug that has been manufactured for the treatment of a disease, disorder, or condition.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies. It is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example. As used herein, “humanized” antibody refers to forms of non-human (e.g. murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity.

“Patient response” or “response” can be assessed using any endpoint indicating a benefit to the patient, including, without limitation, (1) inhibition, to some extent, of disease progression, including stabilization, slowing down and complete arrest; (2) reduction in the number of disease episodes and/or symptoms; (3) inhibition (i.e., reduction, slowing down or complete stopping) of a disease cell infiltration into adjacent peripheral organs and/or tissues; (4) inhibition (i.e. reduction, slowing down or complete stopping) of disease spread; (5) decrease of an autoimmune condition; (6) favorable change in the expression of a biomarker associated with the disorder; (7) relief, to some extent, of one or more symptoms associated with a disorder; (8) increase in the length of disease-free presentation following treatment; or (9) decreased mortality at a given point of time following treatment.

The terms “polypeptide”, “oligopeptide”, “peptide” and “protein” are used interchangaeably herein to refer to chains of amino acids of any length. Peptide include short peptides (e.g., peptides comprising between 2-14 amino acids), medium length peptides (e.g., 15-50) or long chain peptides (e.g., proteins). The chain may be linear or branched, it may comprise modified amino acids, and/or may be interrupted by non-amino acids. Synthetic peptides can be synthesized, for example, using an automated peptide synthesizer. Peptides can also be synthesized by other means such as by cells, bacteria, yeast or other living organisms. Peptides may contain amino acids other than the 20 gene-encoded amino acids. Peptides include those modified either by natural processes, such as processing and other post-translational modifications, but also by chemical modification techniques. Such modifications are well described in basic texts and in more detailed monographs, and are well-known to those of skill in the art. Modifications occur anywhere in a peptide, including the peptide backbone, the amino acid side chains, and the amino or carboxyl termini. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. Also, an antibody “specifically binds” or “preferentially binds” to a target if it binds with greater affinity, avidity, more readily, and/or with greater duration to that target in a sample than it binds to other substances present in the sample.

As used herein, a “pharmaceutically acceptable carrier” or “therapeutic effective carrier” is aqueous or nonaqueous (solid), for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, and oils such as olive oil. A pharmaceutically acceptable carrier can contain physiologically acceptable compounds that act, for example, to stabilize or to increase the absorption of specific inhibitor, for example, carbohydrates, such as glucose, sucrose or dextrans, antioxidants such as ascorbic acid or glutathione, chelating agents, low molecular weight proteins or other stabilizers or excipients.

The “therapeutic compounds” disclosed herein refer to small molecules, chemical entities, nucleic acids, nucleic acid derivatives, peptides, peptide derivatives, naturally-. occurring proteins, non-naturally-occurring proteins, and glycoproteins that are administered to subjects to treat necrotizing enterocolitis. Non-limiting examples of therapeutic compounds include polypeptides such as Mfge8, antibodies, antibody fragments, or antibody derivatives. Exemplary therapeutic compounds include small molecules, chemical entities, nucleic acids, nucleic acid derivatives, peptides, peptide derivatives, naturally-occurring proteins, non-naturally-occurring proteins, peptide-nucleic acids (PNA), stapled peptides, oligonucleotides, morpholinos, antisense drugs, RNA-based silencing drugs, aptamers, and glycoproteins. The term “therapeutic compound” as used herein has essentially the same meaning as the terms “drug” or “therapeutic agent.”

As used herein, “treatment” refers to clinical intervention in an attempt to alter the natural course of the individual or cell being treated and can be performed before or during the course of clinical pathology. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, and achieving remission or improved prognosis. In some embodiments, methods and compositions of the invention are useful in attempts to delay development of a disease or disorder.

Human Mfge8 (lactadherin) contains an integrin binding EGF2 domain (SEQ ID NO:1 at amino acid Nos. 25-68). It also contains two discoidin domains: DD1 (SEQ ID NO:1 at amino acid Nos. 69-227) and DD2 (SEQ ID NO:1 at amino acid Nos. 229-387). Mfge8 affects apoptotic cell clearance, collagen resorption, fatty acid uptake and smooth muscle contraction through the binding EGF2 domain and at least part of one of the 2 discoidin domains. (Borisenko, G G., Cell Death and Differentiation, 11:943-945 (2004); Atabai, K., J. Clin. Investigation, 119(12):3713-3722 (2009); Soltani, A., Nature Med., 20(2):142-153 (2014); Kudo, M., Proc. Nat'l. Acad. Sci. USA, 110(2):660-665 (2013), each of which are incorporated by reference herein in their entirety).

In human Mfge8, the RGD site at position 46-48 of SEQ ID NO:1 participates in integrin binding. Thus, some embodiments of the invention contemplate Mfge8 variants or truncations that maintain the RGD site or its functional equivalent. In other embodiments, the invention contemplates maintaining or enhancing the RGD functionality through conservative or non-conservative amino acid substitutions along the Mfge8 peptide backbone. In other embodiments, the RGD functionality is maintained where certain amino acid substitutions improve the Mfge8 stability profile.

In some embodiments, the Mfge8 protein contains an amino acid substitution. In certain aspects, an amino acid substitution may be a substitution of a residue to any other residue. In certain embodiments, an amino acid substitution includes a conservative amino acid substitution, wherein a residue is replaced with a residue of similar charge or other property. In other embodiments, an amino acid substitution includes a non-conservative amino acid substitution wherein a residue is replaced with a residue that does not have similar charge or other property. An amino acid substitution can be a substitution of a residue to a residue selected from: A, C, D, F, F, G, H, I, K, L, M, N, P, Q, R, S, T, V, W or Y. When an Mfge8 polypeptide includes more than one amino acid substitution, the substitutions may include any one or any combination of the foregoing amino acid substitutions. For example, all of the substitutions may be conservative amino acid substitutions or may be non-conservative amino acid substitutions. Alternatively, the amino acid substitutions may include any combination, such as, for example, one conservative amino acid substitution, and one non-conservative amino acid substitution.

The invention contemplates using a wild-type Mfge8 protein. Other embodiments use the sequence of any one of SEQ ID NOS:1-7 but with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 amino acid substitutions. Other embodiments use proteins that retain all or part of the Mfge8 protein function and are homologous to any one of SEQ ID NOS:1-7. These homologs may be about 85%, 86%, 87%, 88%, 89%, 90%, 91%, 92%, 93%, 94%, 95%, 96%, 97%, 98%, or 99% identical to any one of SEQ ID NOS:1-7.

Mfge8 may be provided in the form of synthesized peptides. Peptide synthesis and recombinant expression are known in the art. The peptides of the invention may be synthesized chemically or biologically, and can include cysteine-rich peptides, circular peptides, stapled peptides, peptides that include D- or L-amino acids and mixtures thereof, peptidomimetics, peptide-nucleic acids (PNAs), and combinations thereof.

Also contemplated within the scope of embodiments described herein are therapeutic peptides that are branched or cyclic, with or without branching. Cyclic, branched and branched circular peptides result from post-translational natural processes and are also made by suitable synthetic methods. In some embodiments, any peptide product described herein comprises a peptide analog described above that is then covalently attached to an alkyl-glycoside surfactant moiety.

Other embodiments include therapeutic peptide chains that are comprised of natural and unnatural amino acids or analogs of natural amino acids. As used herein, peptide and/or protein “analogs” comprise non-natural amino acids based on natural amino acids, such as tyrosine analogs, which includes para-substituted tyrosines, ortho-substituted tyrosines, and meta-substituted tyrosines, wherein the substituent on the tyrosine comprises an acetyl group, a benzoyl group, an amino group, a hydrazine, an hydroxyamine, a thiol group, a carboxy group, a methyl group, an isopropyl group, a C2-C20 straight chain or branched hydrocarbon, a saturated or unsaturated hydrocarbon, an O-methyl group, a polyether group, a halogen, a nitro group, or the like.

Additional embodiments include therapeutic peptide chains having modified amino acids. Examples include acylated amino acids at the ε-position of Lysine, amino acids with fatty acids such as octanoic, decanoic, dodecanoic, tetradecanoic, hexadecanoic, octadecanoic, 3-phenylpropanoic acids and the like, or with saturated or unsaturated alkyl chains. (Zhang, L. and Bulaj, G. (2012) Curr Med Chem 19: 1602-1618, incorporated herein by reference in its entirety).

The invention further contemplates therapeutic peptide chains comprising natural and unnatural amino acids or analogs of natural amino acids. In some embodiments, peptide or protein “analogs” comprise non-natural amino acids based on natural amino acids, such as tyrosine analogs, which includes para-substituted tyrosines, ortho-substituted tyrosines, and meta-substituted tyrosines, wherein the substituent on the tyrosine comprises an acetyl group, a benzoyl group, an amino group, a hydrazine, an hydroxyamine, a thiol group, a carboxy group, a methyl group, an isopropyl group, a C2-C20 straight chain or branched hydrocarbon, a saturated or unsaturated hydrocarbon, an O-methyl group, a polyether group, a halogen, a nitro group, or the like. Examples of Tyr analogs include 2,4-dimethyl-tyrosine (Dmt), 2,4-diethyl-tyrosine, 4-propyl-tyrosine, Ca-methyl-tyrosine and the like. Examples of lysine analogs include ornithine (Orn), homo-lysine, Ca-methyl-lysine (CMeLys), and the like. Examples of phenylalanine analogs include, but are not limited to, meta-substituted phenylalanines, wherein the substituent comprises a methoxy group, a C1-C20 alkyl group, for example a methyl group, an allyl group, an acetyl group, or the like. Specific examples include, but are not limited to, 2,4,6-trimethyl-L-phenylalanine (Tmp), O-methyl-tyrosine, 3-(2-naphthyl)alanine (Nal(2)), 3-(1-naphthyl)alanine (Nal(1)), 3-methyl-phenylalanine, 1,2,3,4-tetrahydroisoquinoline-3-carboxylic acid (Tic), fluorinated phenylalanines, isopropyl-phenylalanine, p-azido-phenylalanine, p-acyl-phenylalanine, p-benzoyl-phenylalanine, p-iodo-phenylalanine, p-bromophenylalanine, p-amino-phenylalanine, and isopropyl-phenylalanine, and the like.

Also contemplated within the scope of embodiments are therapeutic peptide chains containing nonstandard or unnatural amino acids known to the art, for example, C-alpha-disubstituted amino acids such as Aib, Ca-diethylglycine (Deg), aminocyclopentane-1-carboxylic acid (Ac4c), aminocyclopentane-1-carboxylic acid (Ac5c), and the like. Such amino acids frequently lead to a restrained structure, often biased toward an alpha helical structure (Kaul, R, and Balaram, P. (1999) Bioorg Med Chem 7: 105-117, incorporated herein by reference in its entirety). Additional examples of such unnatural amino acids useful in analog design are homo-arginine (Har) and the like. Substitution of reduced amide bonds in certain instances leads to improved protection from enzymatic destruction or alters receptor binding. By way of example, incorporation of a Tic-Phe dipeptide unit with a reduced amide bond between the residues (designated as Tic-F[CH2-NH]∧-Phe) reduces enzymatic degradation.

In some embodiments, modifications at the amino or carboxyl terminus may optionally be introduced into the present peptides or proteins (Nestor, J. J., Jr. (2009) Current Medicinal Chemistry 16: 4399-4418). For example, the present peptides or proteins can be truncated or acylated on the N-terminus (Gourlet, P., et al. (1998) Eur J Pharmacol 354: 105-1 1 1, Gozes, I. and Furman, S. (2003) Curr Pharm Des 9: 483-494), the contents of which is incorporated herein by reference in their entirety). Other modifications to the N-terminus of peptides or proteins, such as deletions or incorporation of D-amino acids such as D-Phe result in potent and long acting agonists or antagonists when substituted with the modifications described herein such as long chain alkyl glycosides.

Thus, the invention provides therapeutic compound analogs wherein the native therapeutic compound is modified by acetylation, acylation, PEGylation, ADP-ribosylation, amidation, covalent attachment of a lipid or lipid derivative, covalent attachment of phosphotidylinositol, cross-linking, cyclization, disulfide bond formation, demethylation, formation of covalent cross-link formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, (Nestor, J. J., Jr. (2007) Comprehensive Medicinal Chemistry II 2: 573-601, Nestor, J. J., Jr. (2009) Current Medicinal Chemistry 16: 4399-4418, Uy, R. and Wold, F. (1977) Science 198:890-6, Seifter, S. and Englard, S. (1990) Methods Enzymol. 182: 626-646, Rattan, S. I., et al. (1992) Ann. NY Acad Sci 663: 48-62). The foregoing references are incorporated by reference in their entirety.

Glycosylated therapeutic peptides may be prepared using conventional Fmoc chemistry and solid phase peptide synthesis techniques, e.g., on resin, where the desired protected glycoamino acids are prepared prior to peptide synthesis and then introduced into the peptide chain at the desired position during peptide synthesis. Thus, the therapeutic peptide polymer conjugates may be conjugated in vitro. The glycosylation may occur before deprotection. Preparation of amino acid glycosides is described in U.S. Pat. No. 5,767,254, WO 2005/097158, and Doores, K., et al., Chem. Commun., 1401-1403, 2006, which are incorporated herein by reference in their entirety. For example, alpha and beta selective glycosylations of serine and threonine residues are carried out using the Koenigs-Knorr reaction and Lemieux's in situ anomerization methodology with Schiff base intermediates. Deprotection of the Schiff base glycoside is then carried out using mildly acidic conditions or hydrogenolysis. A composition, comprising a glycosylated therapeutic peptide conjugate is made by stepwise solid phase peptide synthesis involving contacting a growing peptide chain with protected amino acids in a stepwise manner, wherein at least one of the protected amino acids is glycosylated, followed by water-soluble polymer conjugation. Such compositions may have a purity of at least 95%, at least 97%, or at least 98%, of a single species of the glycosylated and conjugated therapeutic peptide.

Monosaccharides that may be used for introduction at one or more amino acid residues of the therapeutic peptides defined and/or disclosed herein include glucose (dextrose), fructose, galactose, and ribose. Additional monosaccharides suitable for use include glyceraldehydes, dihydroxyacetone, erythrose, threose, erythrulose, arabinose, lyxose, xylose, ribulose, xylulose, allose, altrose, mannose, N-Acetylneuraminic acid, fucose, N-Acetylgalactosamine, and. N-Acetylglucosamine, as well as others. Glycosides, such as mono-, di-, and trisaccharides for use in modifying a therapeutic peptide, one or more amino acid residues of the therapeutic peptides defined and/or disclosed herein include sucrose, lactose, maltose, trehalose, melibiose, and cellobiose, among others. Trisaccharides include acarbose, raffinose, and melezitose.

In further embodiments of the invention, the therapeutic compounds defined and/or disclosed herein may be chemically coupled to biotin. The biotin/therapeutic compound can then bind to avidin.

Another aspect includes recombinant or synthesized Mfge8 variant compositions. In certain embodiments, recombinant Mfge8 variant compositions comprise cell-derived, purified Mfge8 variants. In other embodiments, human Mfge8 variant precursor proteins are purified from an in vitro transfected cell culture.

In certain embodiments, a variant Mfge8 polypeptide comprises post-translational modifications. Exemplary post-translational protein modifications include phosphorylation, acetylation, methylation, ADP-ribosylation, ubiquitination, glycosylation, carbonylation, sumoylation, biotinylation, lipidation, or addition of a polypeptide side chain or of a hydrophobic group. Effects of such non-amino acid elements on the functionality of an Mfge8 may be tested for its biological activity, for example, its ability to bind FeRn.

In certain embodiments, an Mfge8 variant polypeptide may be conjugated to a non-protein agent. Such non-protein agents include, but are not limited to, nucleic acid molecules, chemical agents, organic molecules, etc., each of which may be derived from natural sources, such as for example natural product screening, or may be chemically synthesized.

In certain embodiments, at least one of said amino acid substitutions in an Mfge8 variant is conserved across multiple species. In certain embodiments, a plurality of said amino acid substitutions in an Mfge8 variant are of residues that are conserved across multiple species. In certain embodiments, at least one of said amino acid substitutions in an Mfge8 variant is of a residue that is conserved among serum Mfge8 proteins from human, pig, rat, mouse, dog, rabbit, cow, chicken, donkey, sheep, cat, and horse. In certain embodiments, a plurality of said amino acid substitutions in an Mfge8 variant are of residues that are conserved among serum Mfge8 proteins from human, pig, rat, mouse, dog, rabbit, cow, chicken, donkey, sheep, cat, and horse.

Another aspect includes a protein fusion comprising an Mfge8 variant polypeptide and one or more fusion domains, such as immunoglobulin domains, polyhistidine, Glu-Glu, glutathione S transferase (GST), thioredoxin, protein A, protein G, an immunoglobulin heavy chain constant region (Fe), or maltose binding protein (MBP), which may be used for isolation of the fusion protein by affinity chromatography. For the purpose of affinity purification, relevant matrices for affinity chromatography, such as glutathione-, amylase-, and nickel- or cobalt-conjugated resins are used. Fusion domains also include “epitope tags,” which are usually short peptide sequences for which a specific antibody is available. Useful epitope tags include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation.

In some embodiments, modifications at the amino or carboxyl terminus may optionally be introduced into an Mfge8 variant polypeptide. For example, an Mfge8 variant polypeptide can be truncated or acylated on the N-terminus.

### Mfge8 Variant Expression Systems

In certain embodiments, the recombinant nucleic acids encoding an Mfge8 variant polypeptide may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding an Mfge8 variant polypeptide operably linked to at least one regulatory sequence. Regulatory sequence for use herein include promoters, enhancers, and other expression control elements. In certain embodiments, an expression vector is designed considering the choice of the host cell to be transformed, the particular Mfge8 variant polypeptide desired to be expressed, the vector's copy number, the ability to control that copy number, or the expression of any other protein encoded by the vector, such as antibiotic markers.

Another aspect includes screening gene products of combinatorial libraries generated by the combinatorial mutagenesis of a nucleic acid described herein. Such screening methods include, for example, cloning the gene library into replicable expression vectors, transforming appropriate cells with the resulting library of vectors, and expressing the combinatorial genes under conditions to form such library. The screening methods optionally further comprise detecting a desired activity and isolating a product detected, Each of the illustrative assays described below are amenable to high-throughput analysis as necessary to screen large numbers of degenerate sequences created by combinatorial mutagenesis techniques.

Certain embodiments include expressing a nucleic acid in microorganisms. One embodiment includes expressing a nucleic acid in a bacterial system, for example, in Bacillus brevis, Bacillus megaterium, Bacillus subtilis, Caulobacter crescentus, Escherichia coli and their derivatives. Exemplary promoters include the 1-arabinose inducible araBAD promoter (PBAD), the lac promoter, the 1-rhamnose inducible rhaP BAD promoter, the T7 RNA polymerase promoter, the trc and tac promoter, the lambda phage promoter Pl, and the anhydrotetracycline-inducible tetA promoter/operator.

Other embodiments include expressing a nucleic acid in a yeast expression system. Exemplary promoters used in yeast vectors include the promoters for 3-phosphoglycerate kinase (Hitzeman et al., J. Biol. Chem. 255:2073 (1980)); other glycolytic enzymes (Hess et al., J. Adv. Enzyme Res. 7:149 (1968); Holland et al., Biochemistry 17:4900 (1978). Others promoters are from, e.g., enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, glucokinase alcohol oxidase I (AOX1), alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter and termination sequences, with or without an origin of replication, is suitable. Certain yeast expression systems are commercially available, for example, from Clontech Laboratories, Inc. (Palo Alto, Calif., e.g. Pyex 4T family of vectors for S. cerevisiae), Invitrogen (Carlsbad, Calif., e.g. Ppicz series Easy Select Pichia Expression Kit) and Stratagene (La Jolla, Calif., e.g. ESP.TM. Yeast Protein Expression and Purification System for S. pombe and Pesc vectors for S. cerevisiae).

Other embodiments include expressing a nucleic acid in mammalian expression systems. Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In a specific embodiment, a yeast alcohol oxidase promoter is used.

In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273: 113-120 (1978). The immediate early promoter of the human cytomegalovirus is conveniently obtained as a HindIII E restriction fragment. Greenaway, P. J. et al., Gene 18: 355-360 (1982). The foregoing references are incorporated by reference in their entirety.

Other embodiments include expressing a nucleic acid in insect cell expression systems. Eukaryotic expression systems employing insect cell hosts may rely on either plasmid or baculoviral expression systems. Typical insect host cells are derived from the fall army worm (Spodoptera frugiperda). For expression of a foreign protein these cells are infected with a recombinant form of the baculovirus Autographa californica nuclear polyhedrosis virus which has the gene of interest expressed under the control of the viral polyhedron promoter. Other insects infected by this virus include a cell line known commercially as “High 5” (Invitrogen) which is derived from the cabbage looper (Thichoplusia ni). Another baculovirus sometimes used is the Bombyx mori nuclear polyhedorsis virus which infect the silk worm (Bombyx mori). Numerous baculovirus expression systems are commercially available, for example, from Thermo Fisher (Bac-N-Blue™ k or BAC-TO-BAC™ Systems), Clontech (BacPAK™ Baculovirus Expression System), Novagen (Bac Vector System™), or others from Pharmingen or Quantum Biotechnologies. Another insect cell host is the common fruit fly, Drosophila melanogaster, for which a transient or stable plasmid based transfection kit is offered commercially by Thermo Fisher (The DES™ System).

In some embodiments, cells are transformed with vectors that express a nucleic acid described herein. Transformation techniques for inserting new genetic material into eukaryotic cells, including animal and plant cells, are well known. Viral vectors may be used for inserting expression cassettes into host cell genomes. Alternatively; the vectors may be transfected into the host cells. Transfection may be accomplished by calcium phosphate precipitation, electroporation, optical transfection, protoplast fusion, impalefection, and hydrodynamic delivery.

Certain embodiments include expressing a nucleic acid encoding an Mfge8 variant polypeptide in mammalian cell lines, for example Chinese hamster ovary cells (CHO) and Vero cells. The method optionally further comprises recovering the Mfge8 variant polypeptide.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

In some embodiments, MFGE8, or an integrin binding domain thereof, is fused to an antibody or an immunoglobulin domain. In other embodiments, the RGD integrin binding domain is fused to an antibody or immunoglobulin domain. In preferred embodiments, the antibody or immunoglobulin domain is an immunoglobulin A (IgA) domain.

The term antibody is meant to include monoclonal antibodies, polyclonal antibodies, humanized antibodies, antibody fragments (e.g., Fe domains), Fab fragments, single chain antibodies, bi- or multi-specific antibodies, Llama antibodies, nano-bodies, diabodies, affibodies, Fv, Fab, F(ab′)2, Fab′, scFv, scFv-Fc, and the like. Also included in the term are antibody-fusion proteins, such as Ig chimeras. Preferred antibodies include humanized or fully human monoclonal antibodies or fragments thereof.

The terms “full length antibody,” “intact antibody” and “whole antibody” are used herein interchangeably to refer to an antibody in its substantially intact form, not antibody fragments as defined below. The terms particularly refer to an antibody with heavy chains that contain the Fc region. “Antibody fragments” comprise a portion of an intact antibody, preferably comprising the antigen binding region thereof. Examples of antibody fragments include Fab, Fab′, F(ab′)2, and Fv fragments; diabodies; linear antibodies; single-chain antibody molecules; and multispecific antibodies formed from antibody fragments. The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies.

In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations that typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

Antibodies that bind specifically to an antigen have a high affinity for that antigen. Antibody affinities may be measured by a dissociation constant (Kd). In certain embodiments, an antibody provided herein has a dissociation constant (Kd) of equal to or less than about 100 nM, 10 nM, 1 nM, 0.1 nM, 0.01 nM, or 0.001 nM (e.g. 10⁻⁷ M or less, from 10⁻⁷ M to 10⁻¹³ M, from 10⁻⁸ M to 10⁻¹³ M or from 10⁻⁹ M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [125I]-antigen are mixed with serial dilutions of a Fab of interest (e.g., consistent with assessment of the anti-VEGF antibody, Fab-12, in Presta et at, Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with, e.g., immobilized antigen CMS chips at ˜10 response units (RU), Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (Kₒₙ) and dissociation rates (K_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. Other coupling chemistries for the target antigen to the chip surface (e.g., streptavidin/biotin, hydrophobic interaction, or disulfide chemistry) are also readily available instead of the amine coupling methodology (CMS chip) described above, as will be understood by one of ordinary skill in the art.

The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including, for example, the hybridoma method (e.g., Kohler et al, Nature, 256: 495 (1975); Harlow et al., Antibodies: A Laboratory Manual, (Cold Spring Harbor Laboratory Press, 2nd ed. 1988); Hammerling et al., Monoclonal Antibodies and T-Cell Hybridomas pp. 563-681 (Elsevier, N.Y., 1981)), recombinant DNA methods (see, e.g., U.S. Pat. No. 4,816,567), phage display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immununol. Methods 284(1-2): 119-132(2004), and technologies for producing human or human-like antibodies in animals that have parts or all of the human immunoglobulin loci or genes encoding human immunoglobulin sequences (see, e.g., W098/24893; WO96/34096; W096/33735; WO91/10741; Jakobovits et al., Proc. Natl. Acad. Sci, USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; 5,661,016; Marks et al., Bio. Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996) and Lonberg and Huszar, Intern. Rev Immunol. 13: 65-93 (1995). The above patents, publications, and references are incorporated by reference in their entirety.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity; affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fe), typically that of a human immunoglobulin. For further details, see Jones et al., Nature 321:522-525 (1986); Riechmann et al., Nature 332:323-329 (1988); and Presta, Curr Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23: 1035-1038 (1995); Hurle and Gross, Curr. Op. Biotech. 5:428-433 (1994). The foregoing references are incorporated by reference in their entirety.

A “human antibody” is one which comprises an amino acid sequence corresponding to that of an antibody produced by a human and/or has been made using any of the techniques for making human antibodies as disclosed herein. Such techniques include screening human-derived combinatorial libraries, such as phage display libraries (see, e.g., Marks et al., J. Mol. Biol, 222: 581-597 (1991) and Hoogenboom et al., Nucl. Acids Res., 19: 4133-4137 (1991)); using human myeloma and mouse-human heteromyeloma cell lines for the production of human monoclonal antibodies (see, e.g., Kozbor, J. Immunol, 133: 3001 (1984); Brodeur et al., Monoclonal Antibody Production Techniques and Applications, pp. 55-93 (Marcel Dekker, Inc., New York, 1987); and Boerner et al., J. Immunol, 147: 86 (1991)); and generating monoclonal antibodies in transgenic animals (e.g., mice) that are capable of producing a full repertoire of human antibodies in the absence of endogenous immnunoglobulin production (see, e.g., Jakobovits et al., Proc. Natl. Acad. Sci USA, 90: 2551 (1993); Jakobovits et al., Nature, 362: 255 (1993); Bruggermann et al., Year in Immunol., 7: 33 (1993)). This definition of a human antibody specifically excludes a humanized antibody comprising antigen-binding residues from a non-human animal.

Pharmaceutical compositions of this invention comprise any of the compounds of the present invention, and pharmaceutically acceptable salts thereof, with any pharmaceutically acceptable carrier, adjuvant or vehicle. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts retain the desired biological activity of the therapeutic composition without toxic side effects. Examples of such salts are (a) acid addition salts formed with inorganic acids, for example, hydrochloric acid, hydrobromic acid, sulfuric acid, phosphoric acid, nitric acid and the like/and salts formed with organic acids such as, for example, acetic acid, trifluoroacetic acid, tartaric acid, succinic acid, maleic acid, fumaric acid, gluconic acid, citric acid, malic acid, ascorbic acid, benzoic acid, tanic acid, pamoic acid, alginic acid, polyglutamic acid, naphthalenesulfonic acid, naphthalene disulfonic acid, polygalacturonic acid and the like; (b) base addition salts or complexes formed with polyvalent metal cations such as zinc, calcium, bismuth, barium, magnesium, aluminum, copper, cobalt, nickel, cadmium, and the like; or with an organic cation formed from N,N′-dibenzylethylenediamine or ethlenediamine; or (c) combinations of (a) and (b), e.g. a zinc tannate salt and the like.

The pharmaceutical compositions of this invention may be administered orally. They may contain any conventional, non-toxic, pharmaceutically-acceptable carriers, adjuvants or vehicles. Also contemplated, in some embodiments, are pharmaceutical compositions comprising as an active ingredient, therapeutic compounds described herein, or pharmaceutically acceptable salt thereof, in a mixture with a pharmaceutically acceptable, non-toxic component. The compositions may conveniently be administered in unit dosage form and may be prepared by any of the methods well-known in the pharmaceutical art, for example, as described in Remington Pharmaceutical Sciences, 17th ed., Mack Publishing Co., Easton, Pa. (1985), incorporated herein by reference in its entirety.

The pharmaceutical compositions of this invention may be orally administered in any orally acceptable dosage form including, but not limited to, capsules, tablets, and aqueous suspensions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Additionally, the pharmaceutical compositions may be administered in milk, formulae, yogurt, juice, or other common food or beverages known in the art.

The pharmaceutical compositions of this invention may also be administered in the form of suppositories for rectal administration. These compositions can be prepared by mixing a compound of this invention with a suitable non-irritating excipient that is solid at room temperature but liquid at the rectal temperature and therefore will melt in the rectum to release the active components. Such materials include, but are not limited to, cocoa butter, beeswax and polyethylene glycols.

The invention contemplates dosage levels of between about 0.001 and about 100 mg Mfge8/kg body weight per day, preferably between about 0.005 and about 50 mg/kg, 0.01 and about 10 mg/kg, 0.05 and about 5 mg/kg, 0.1 and about 1 mg/kg body weight. Other embodiments contemplate a dosage of between about 0.001-0.010, 0.010-0.050, 0.050-0.100, 0.1-0.5, 0.5-1.0, 1.0-5.0, 5.0-10, or 10-50 mg/kg body weight. The dosages may be administered about hourly, postprandial, daily, with every meal, every other day, weekly, monthly, or on an as-needed basis

Such administration can be used as a chronic or acute therapy. The amount of drug that may be combined with the carrier to produce a single dosage form will vary depending upon the host treated and the particular mode of administration. A typical preparation will contain from about 5% to about 95% active compound (w/w). Preferably, such preparations contain from about 20% to about 80%, 30% to about 70%, 40% to about 60%, or about 50% active compound. In other embodiments, the preparations used in the invention will be about 5-10%, 10-20%, 20-30%, 30-40%, 40-50%, 50-60%, 60-70%, 70-80%, 80-90%, 90-99%, or greater than 99% of the active ingredient.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

As the skilled artisan will appreciate, lower or higher doses than those recited above may be required. Specific dosage and treatment regimens for any particular patient will depend upon a variety of factors, including the activity of the specific compound employed, the age, body weight, general health status, gender, diet, time of administration, rate of excretion, drug combination, the severity and course of an infection, the patient's disposition to the infection and the judgment of the treating physician.

In order that the invention described herein may be more fully understood, the following examples are set forth. It should be understood that these examples are for illustrative purposes only and are not to be construed as limiting this invention in any manner.

### EXAMPLES

### Example 1

### TNBS-Induced Murine Model for NEC

To induce enterocolitis, 10-day old C57/B6 mice were given 2,4,6-Trinitrobenzene-sulfonic acid (TNBS) (n=10/group) by gavage and enema. Mice were anesthetized in an isoflurane chamber, a 3.5 gauge French silicone catheter was inserted into the stomach, gastric contents removed, and TNBS (50 mg/kg body weight dissolved in 30% w/v ethanol) was administered by gavage. The catheter was then inserted per rectum to a length of 1-2 cm, and another equal dose administered slowly by enema. These doses were optimized for survival. Control animals were given vehicle alone. Cohorts given rMfge8 received daily doses by oral gavage 11 day post-TNBS treatment. Weight and survival were observed daily for the duration of the experiment.

### Example 2

### rMfge8 Promotes Weight Gain in NEC Model

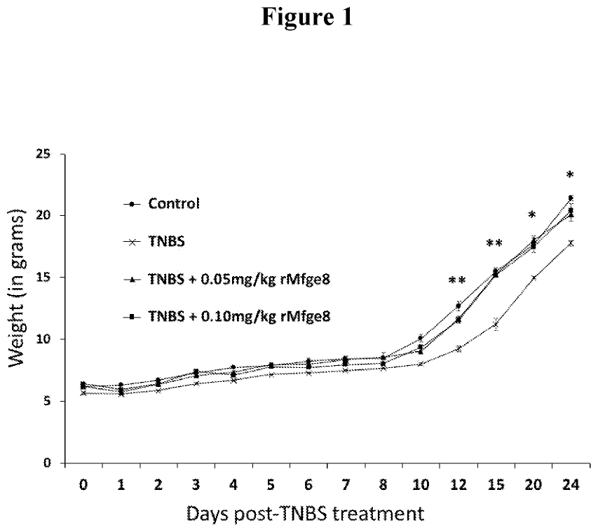

NEC was induced in mice with TNBS as described in Example 1. Control mice were given vehicle alone and thus did not develop NEC. Other cohorts were induced to NEC and subsequently given recombinant mouse Mfge8 at a dose of 0.05 or 0.10 mg/kg once daily by oral gavage. The mouse Mfge8 included amino acids 23-263 of SEQ ID NO:8 plus a C-terminal 6-histidine tag (R&D Systems, Minneapolis, Minn., Catalog No: 2805-MF/CF.) As shown in FIG. 1, the NEC mice showed a considerable weight loss when compared to the control mice. By day 24, the control mice weighed on average 21.4 g. The NEC-induced mice weighed on average 17.8 g, more than 17% less. rMfge8 rescued the weight loss at both the 0.05 (average weight 20.1 g) and 0.10 mg/kg (average weight 20.43 g) dosage. rMfge8 thus rescued the mice to an average of about 94%-95% of the control mice.

### Example 3

### rMfge8 Promotes Neonatal Survival in NEC

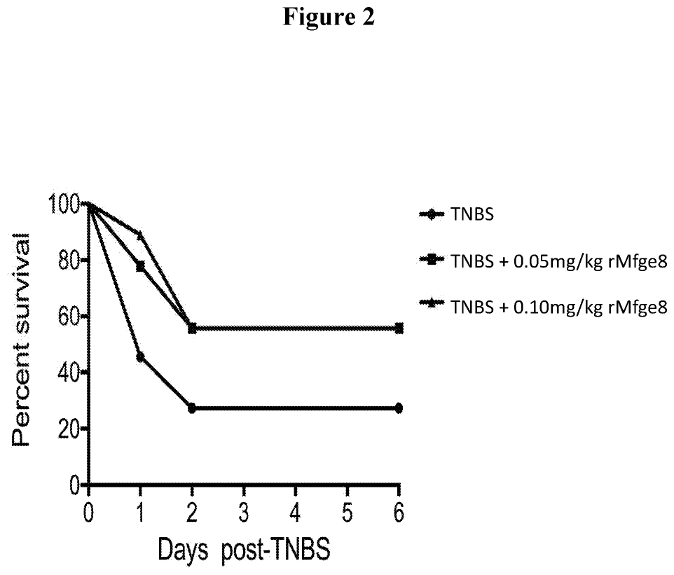

NEC was induced in mice with TNBS as described in Example 1. Control mice were given vehicle alone and thus did not develop NEC. Other cohorts were induced to NEC and subsequently given recombinant Mfge8 at a dose of 0.05 or 0.10 mg/kg once daily by oral gavage. As shown in FIG. 2, After two days, only 27.3% of the NEC mice survived when compared to the control mice. In contrast, 55.6% of the NEC mice survived after receiving either 0.05 or 0.10 mg/kg daily rMfge8.

EXEMPLARY SEQUENCES

(Human Mature Lactadherin isoform a preprotein)
SEQ ID NO: 1
MPRPRLLAAL CGALLCAPSL LVALDICSKN PCHNGGLCEE ISQFVRGDVF
PSYTCTCLKG YAGNHCETKC VEPLGLFNGN IANSQIAASS VRVTFLGLQH
WVPELARLNR AGMVNAWTPS SNDDNPWIQV NLLRRMWVTG VVTQGASRLA
SHEYLKAFKV AYSLNGHEFD FIHDVNKKHK EFVGNWNKNA VHVNLFETPV
EAQYVRLYPT SCHTACTLRF ELLGCELNGC ANPLGLKNNS IPDKQITASS
SYKTWGLHLF SWNPSYARLD KQGNENAWVA GSYGNDQWLQ VDLGSSKEVT
GIITQGARNF GSVQFVASYK VAYSNDSANW TEYQDPRTGS SKIFPGNWDN
HSHKKNLFET PILARYVRIL PVAWHNRIAL RLELLGC
(Human Mature Lactadherin isoform b precursor)
SEQ ID NO: 2
MPRPRLLAAI CGALLCAPSL LVALDICSKN PCHNGGLCEE ISQEVRGDVF
PSYTCTCLKG YAGNHCETKC VEPLGMENGN IANSQIAASS VRVTFLGLQH
WVPELARLNR AGMVNAWTPS SNDDNPWIQV NLLRRMWVTG VVTQGASRLA
SHEYLKAFKV AYSLNGHEFD FIHDVNKKHK EFVGNWNKNA VHVNLFETPV
EAQYVRLYPT SCHTACTLRF ELLGCELNGC ANPLGLKNNS IPDKQITASS
SYKTWGLHLF SWNPSYARLD KQGNFNAWVA GSYGNDQWLQ TFPGNWDNHS
HKKNLFETPI LARYVRILPV AWHNRIALRL ELLGC
(Human Mature Lactadherin isoform c)
SEQ ID NO: 3
MWPFPEGGNT IPILHTDICS KNPCHNGGLC EEISQEVRGD VEPSYTCTCL
KGYAGNHCET KCVEPLGLEN GNIANSQIAA SSVRVTFLGL QHWVPELARI
NRAGMVNAWT PSSNDDNPWI QVNLLRRMWV TGVVTQGASR LASHEYLKAF
KVAYSLNGHE FDFIHDVNKK HKEFVGNWNK NAVHVNLFET PVEAQYVRLY
PTSCHTACTL RFELLGGELN GCANPLGLKN NSIPDKQITA SSSYKTWGLH
LFSWNPSYAR LDKQGNENAW VAGSYGNDQW LQVDLGSSKE VTGITTOGAR
NFGSVQFVAS YKVAYSNDSA NWTFYQDPRT GSSKTFPGNW DNHSHKKNLF
ETPILARYVR ILPVAWHNRI ALRLELLGC
(Human Mature Lactadherin isoform d precursor)
SEQ ID NO: 4
MPRPRLLAAL CGALLCAPSL LVALECVEPL GLENGNTANS QIAASSVRVT
FLGLQHWVPE LARLNRAGMV NAWTPSSNDD NPWIQVNLLR RMWVTGVVTQ
GASRLASHEY LKAFKVAYSL NGHEFDFIHD VNKKHKEFVG NWNKNAVHVN
LFETPVEAQY VRLYPTSCHT ACTLRFELLG CELNGCANPL GLKNNSIPDK
QITASSSYKT WGLHLFSWNP SYARLDKQGN FNAWVAGSYG NDQWLQVDLG
SSKEVTGIIT QGARNEGSVQ EVASYKVAYS NDSANWTEYQ DPRTGSSKIF
PGNWDNHSHK KNLFETPILA RIVRILPVAW HNRIALRIEL LGC
(Human Mature Lactadherin isoform e)
SEQ ID NO: 5
MVNAWTPSSN DDNPWIQVNL LRRMWVTGVV TQGASRTASH EYLKAFKVAY
SLNGHEFDFI HDVNKKHKEF VGNWNKNAVH VNLFETPVEA QYVRLYPTSC
HTACTLRFEL LGGELNGCAN PLGLKNNSIP DKQITASSSY KTWGLHLFSW
NPSYARLDKQ GNFNAWVAGS YGNDQWLQVD LGSSKEVTGI ITQGARNFGS
VQFVASYKVA YSNDSANWTE YQDPRTGSSK IFPGNWDNHS HKKNLFETPI
LARYVRILPV AWHNRTALRL ELLGC
(Human Lactadherin isoform X1)
SEQ ID NO: 6
MFLYRVMWPF PEGGNTIPIL HTDICSKNPC HNGGLCEEIS QEVRGDVEPS
YTCTCLKGYA GNHCETKCVE PLGLENGNIA NSOIAASSVR VTFLGLQHWV
PELARLNRAG MVNAWTPSSN DDNPWIQVNL LRRMWVTGVV TQGASRLASH
FYLKAFKVAY SLNGHEFDFT HDVNKKHKEF VGNWNKNAVH VNLFETPVEA
QYVRLYPTSC HTACTLRFEL LGCELNGCAN PLGLKNNSTP DKQITASSSY
KTWGLHLFSW NPSYARLDKQ GNFNAWVAGS YGNDOWLQVD LGSSKEVTGI
ITQGARNFGS VQFVASYKVA YSNDSANWTE YODPRTGSSK IFPGNWDNHS
HKKNLFETPI LARYVRILPV AWHNRIALRL ELLGC
(Human Lactadherin isoform X2)
SEQ ID NO: 7
MPRPRLLAAL CGALLCAPSL LVALDICSKN PCHNGGLCEE ISQEVRGDVF
PSYTCTCLKG YAGNHCFTKC VEPLGLENGN IANSQTAASS VRVTFLGLQH
WVPELARLNR AGMVNAWTPS SNDDNPWIQV NLLRRMWVTG VVTQGASRLA
SHEYLKAFKV AYSLNGHEFD FIHDVNKKHK EFVGNWNKNA VHVNLFETPV
EAQYVRLYPT SCHTACTLRF ELLGCELNAR KADLRRGADD REQ
(Mouse Mfge8)
SEQ ID NO: 8
MQVSRVLAAL CGMLLCASGL FAASGDFCDS SLCLNGGTCL TGQDNDIYCL
CPEGFTGLVC NETERGPCSP NTCYNDAKCI VTIDTQRGDI FTEYICQCPV
GYSGIHCETE TNYYNLDGEY MFTTAVPNTA VPTPAPTPDL SNNLASRGST
QLGMEGGATA DSQISASSVY MGFMGIQRWG PELARLYRTG IVNAWTASNY
DSKPWIQVNL LRKMEVSGVM TQGASRAGRA EYLKTFKVAY SLDGRKFEFI
QDESGGDKFF LGNLDNNSLK VNMFNPTLEA QYIKLITVSC HRGCTLRFEL
LGCELHGCSE PLGLKNNTIP DSQMSASSSY KTWNLRAFGW YTHLGRLDNQ
GKINAWTAQS NSAKEWLQVD LGTQRQVTGI ITQGARDFGH TQYVASYKVA
HSDDGVQWTV YEEQGSSKVF QGNLDNNSHK KNIFEKPFMA RYVEVLPVSW
HNRITLRIEL LGC

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

1. A method for treating or preventing necrotizing enterocolitis (NEC) in a human infant, comprising orally administering an agonist for an αvβ3, αvβ5, or an α8β1 integrin receptor in intestinal enterocytes of said human infant.

2. (canceled)

3. (canceled)

4. The method of claim 1, wherein said agonist is an isolated milk fat globule-EGF factor 8 (Mfge8) protein having Mfge8 or lactadherin activity.

5. The method of claim 4, wherein said Mfge8 has at least a 90% sequence identity to any one of SEQ ID NOS:1-7.

6. The method of claim 5, wherein said Mfge8 has the sequence of any one of SEQ ID NOS:1-7.

7. The method of claim 1, wherein said Mfge8 is administered at a dosage of between about 0.001 and 0.5 mg/kg of body weight.

8. The method of claim 7, wherein said dosage is between about 0.005 and 0.05 mg/kg of body weight.

9. The method of claim 7, wherein said dosage is between about 0.01 and 0.05 mg/kg of body weight.

10. The method of claim 5, wherein said dosage is about 0.05 mg/kg of body weight.

11. The method of claim 5, wherein said dosage is about 0.10 mg/kg of body weight.

12. The method of claim 5, wherein said dosage is about 0.50 mg/kg of body weight.

13. The method of claim 1, wherein said agonist comprises an immunoglobulin domain.

14. The method of claim 1, wherein said agonist comprises an immunoglobulin A (IgA) domain.

15. The method of claim 13, wherein said agonist further comprises Mfge8.

16. The method of claim 13, wherein said agonist is an antibody that binds to said integrin receptor with an equilibrium dissociation constant (K_{D}) of ≤1 pM, ≤10 pM ≤100 pM, ≤1 nM, ≤10 nM, or ≤100 nM.

17. The method of claim 16, wherein said antibody is a monoclonal antibody.

18. The method of claim 17, wherein said antibody is a human monoclonal antibody.

19. The method of claim 17, wherein said antibody is a humanized monoclonal antibody.

20. The method of claim 1, wherein said agonist is administered in a capsule, tablet, gel, or liquid formulation.

21. A use of an agonist for an αvβ3, αvβ5, or α8β1 integrin receptor for treating necrotizing enterocolitis (NEC) in a human infant.

22. The use of claim 21, wherein said agonist comprises an isolated milk fat globule-EGF factor 8 (Mfge8) protein having Mfge8 or lactadherin activity.


SEQUENCE LISTING (9 sequences)

SEQ ID NO: 1 - protein, 387 aa, Homo sapiens
MPRPRLLAALCGALLCAPSLLVALDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 2 - protein, 335 aa, Homo sapiens [sequence omitted]

SEQ ID NO: 3 - protein, 379 aa, Homo sapiens
MWPFPEGGNTIPILHTDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 4 - protein, 343 aa, Homo sapiens
MPRPRLLAALCGALLCAPSLLVALECVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 5 - protein, 275 aa, Homo sapiens
MVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 6 - protein, 385 aa, Homo sapiens
MFLYRVMWPFPEGGNTIPILHTDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 7 - protein, 243 aa, Homo sapiens
MPRPRLLAALCGALLCAPSLLVALDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNARKADLRRGADDREQ

SEQ ID NO: 8 - protein, 463 aa, Mus sp.
MQVSRVLAALCGMLLCASGLFAASGDFCDSSLCLNGGTCLTGQDNDIYCLCPEGFTGLVCNETERGPCSPNPCYNDAKCLVTLDTQRGDIFTEYICQCPVGYSGIHCETETNYYNLDGEYMFTTAVPNTAVPTPAPTPDLSNNLASRCSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 9 - protein, 6 aa, Artificial Sequence
  Description of Artificial Sequence Synthetic 6xHis tag
HHHHHH